(19) United States
(12) Patent Application Publication
(10) Pub. No.: US 2019/0318814 A1
(43) Pub. Date: Oct. 17, 2019

(54) Health Care System using Blockchain

(71) Applicant: Toby Unwin, Utica, OH (US)

(72) Inventor: Toby Unwin, Utica, OH (US)

(21) Appl. No.: 15/950,861

(22) Filed: Apr. 11, 2018

(51) Int. Cl. G16H 10/60; G06F 17/30; H04L 9/06; G06F 21/60

(52) U.S. Cl. CPC G16H 10/60; G06F 21/602; H04L 9/0637; G06F 17/30371

(57) ABSTRACT

The current invention is a health care blockchain system, method and program that can analyze a set of self learning systems, tackling thousands of variations automatically to normalizes data from each provider health care provider and writes it to a private BlockChain that is secure, cryptographic and permission based while still being decentralized. The health care providers will read and write data through the system, translating between normalized and their formats. These configurations are done automatically and are not done by the physicians or the tech systems.

### CROSS-REFERENCES TO RELATED APPLICATIONS (IF ANY)

None

### BACKGROUND OF INVENTION

### Field of the Invention

The present invention is directed to a method of normalizing health care records using a blockchain system.

### Background

There are a number current issues with health care such as “WHY CAN'T YOU GET THAT FROM MY PRIMARY PHYSICIAN?” New offices have to call and have them fax it over. The current office can't use their computer system pull it from theirs as they're different systems and they can't talk to each other. There are thousands of different systems. None of them talk to each other.

This happened due to the Patient Protection and Affordable Care Act. Every health care provider was forced to use electronic records hoping that paper files would move to computers. But the new systems aren't that easy to use, so most providers have electronic and paper systems.

A typical setup is that the patient arrives, fills out a clipboard. The information is typed into the system and then printed out for the doctor. The doctor's hand written notes are typed back in. A summary is printed out for the patient to sign which is scanned back in again.

No common data format was specified so thousands of different system have their own formats. Doctors exchange information by printing it and faxing it thereby turning structured data into unstructured data, waste and inefficiency. 1,000 providers×1,000 providers=1 Million interfaces needed. The problem is generally seen as “Impossible” to solve.

There is still room for improvement in the art.

### SUMMARY OF THE INVENTION

The current invention is a health care system, method and program product that uses self-learning systems, tackling thousands of variations automatically to normalizes data from each provider health care provider and writes it to private BlockChain that is secure, cryptographic and Permission Based while still being decentralized.

The health care providers will read and write data through the system 1, translating between normalized and their formats. These configurations are done automatically and are not done by the physicians or the tech systems.

Given the significant value of these types of cases the monies involved can be substantial.

### BRIEF DESCRIPTION OF THE DRAWINGS

Without restricting the full scope of this invention, the preferred form of this invention is illustrated in the following drawings:

FIG. 1 is a block diagram showing a basic arrangement of a computer system that can run the current invention; and

FIG. 2 is a schematic block diagram of a conceptualized operation of the present invention

FIG. 3 is an example of a blockchain system.

### BRIEF DESCRIPTION OF THE PREFERRED EMBODIMENTS

There are a number of significant design features and improvements incorporated within the invention.

The current invention is a system 1, method and program product that uses blockchain to normalized health care data for use in health care systems.

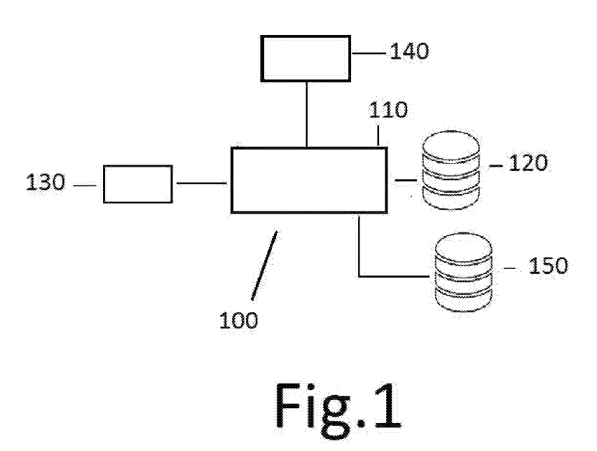

The system 1 can be set up to be run a on a computing device. FIG. 1 is a block diagram showing a computing device 100 on which the present invention can run comprising a CPU 110, Hard Disk Drive 120, Keyboard 130, Monitor 140, CPU Main Memory 150 and a portion of main memory where the program resides and executes. A printer can also be included. Any general purpose computer with an appropriate amount of storage space is suitable for this purpose. Computer Devices like this are well known in the art and is not pertinent to the invention.

The computer device 100 could be connected to other computer devices 100 through a communication interface such as the Internet, a wide area network (WAN), internetwork, telephone network or a private Value Added Network (VAN).

The storage and databases for the system may be implemented by a single data base structure at an appropriate site, or by a distributed data base structure that is distributed across an intra or an Internet network.

The files and file components discussed herein may be paper files, but in a preferred embodiment comprise data structures with electronic data. The setting up of the files and file structure is commonly known in the art and is not disclosed here.

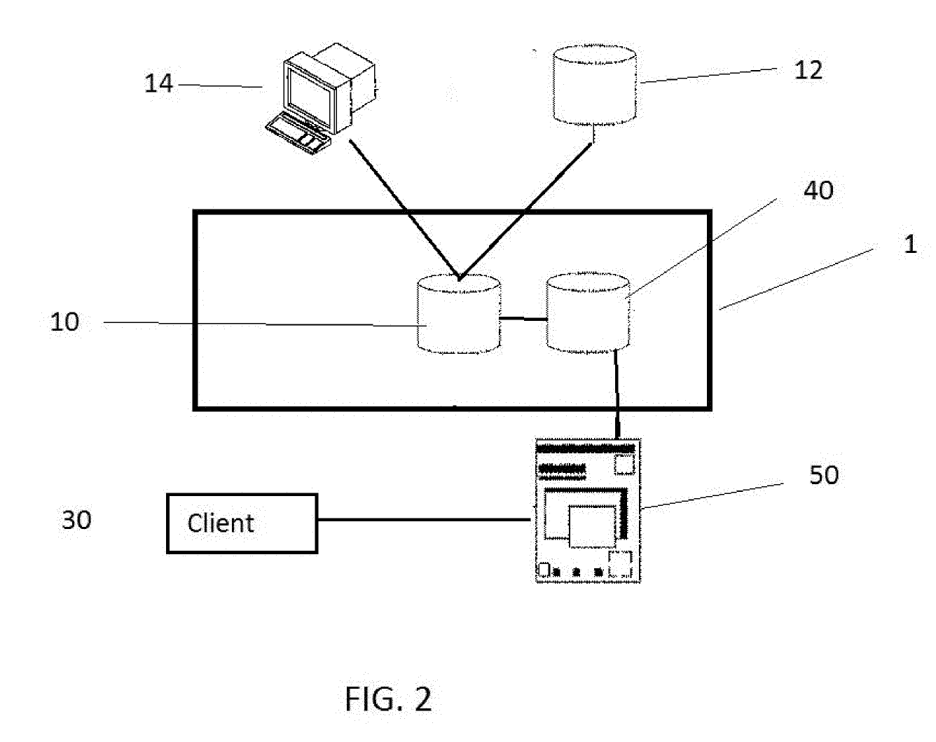

This system 1 is shown in FIG. 2, the System 1 will gather information 10 about a person's health care records.

The information 10 can be gathered from heath care/doctor's/hospital's databases 12 or keyed into the system 1 by a keyboard entry 14 or any other method of entry. The information 10 will have data on everything relevant available that can be used to for the health care of the patient.

This data 40 can be specifically analyzed using a computer processing means for the client 30 depending on their needs or their specific case. The client 30 can be an individual, a small company, an doctor or even a large hospital or insurer.

The data 40 is loaded into electronic medium and is analyzed by the system 1 based on the desired criteria.

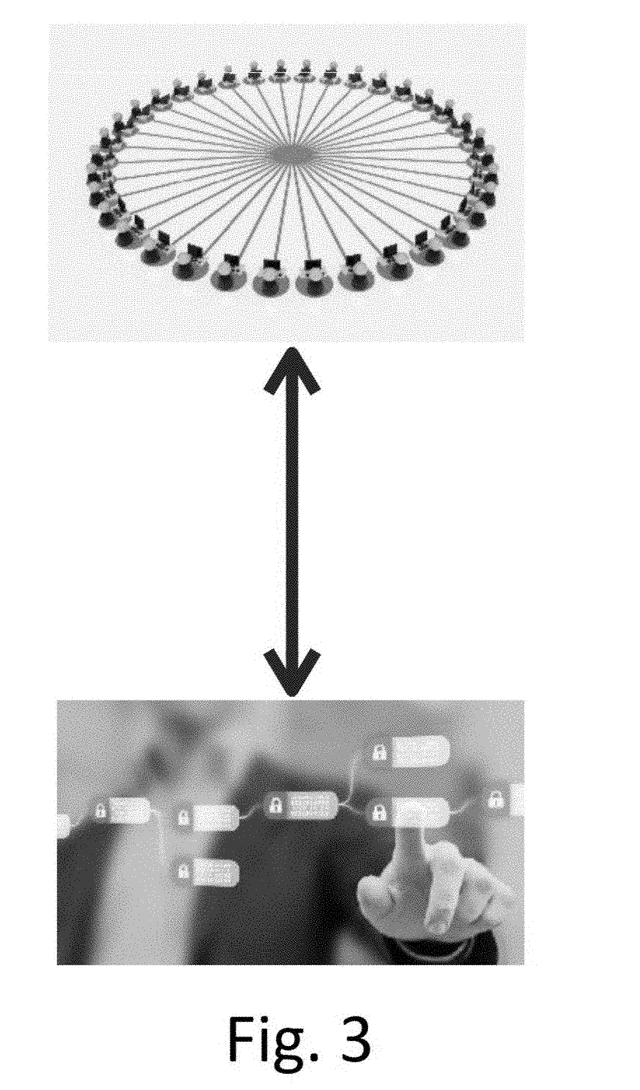

The system 1 will use Self learning systems, tackling thousands of variations automatically. The system 1 normalizes data from each provider and writes it to a private BlockChain, as shown in FIG. 3, that is secure, cryptographic and permission based while still being decentralized.

The health care providers will read and write data through the system 1, translating between normalized and their formats. These configurations are done automatically and are not done by the physicians or the tech systems.

The system 1 can be used by and with any health care provider or insurer like Medicare. The system 1 makes it cheaper for the providers to provide their services saving them money. Insurers are 80%+ of the health care market. Every participant has to deal with them, because they need to get paid.

The system 1 allows for providers to submit records via a normalized BlockChain. This would bring transparency to law and provide billions of dollars in savings.

The system 1 will provide more data that is easier to access and use. The system's A.I. can provide claims review with immediate access to essentially all medical data. The system 1 will find fraud and help identifies which claims can be paid easily producing drastic reductions in payment processing costs.

### Advantages

Health care is a $4 Trillion industry with 21% of GDP which is the Largest of all. 33% of overhead is administrative—caused mainly by the lack of provider communication and currently almost completely lacking in transparency. The system 1 will reduce costs and add transparency.

It should be appreciated that many other similar configurations are within the abilities of one skilled in the art and all of these configurations could be used with the method of the present invention. Furthermore, it should be recognized that the computer system and network disclosed herein can be programmed and configured by one skilled in the art in a variety of different manners to implement the method steps described further herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A method comprising the steps: having a information about health care record, loading this information into a database stored on electric means, normalizing the data, using a decentralized blockchain that is secure, cryptographic and permission based.
